(19) United States
(12) Patent Application Publication
(10) Pub. No.: US 2004/0199235 A1
(43) Pub. Date: Oct. 7, 2004

(54) Electrode system for neural applications

(76) Inventor: Imad Younis, Nazareth Illit (IL)

Correspondence Address: William H Dippert, Reed Smith, 599 Lexington Avenue, 29th Floor, New York, NY, 10022-7650, US

(21) Appl. No.: 10/491,259

(22) PCT Filed:
(86) PCT No.: PCT/IL02/00796

(30) Foreign Application Priority Data
Sep. 30, 2001 (IL) 145700

(51) Int. Cl. A61N 1/05

(52) U.S. Cl. 607/116

(57) ABSTRACT

Methods and apparatus for positioning electrodes in a skull. In some embodiments of the invention multiple measurements are made and a desired location is determined or estimated from the results of the measurements.

### FIELD OF THE INVENTION

The present invention relates to the field of implanting electrodes in the brain, for temporary and/or permanent applications.

### BACKGROUND OF THE INVENTION

Electrodes are implanted deep into the brain for various reasons. One of the newer applications is implanting electrodes in motor regions of a patient to overcome at least some of the symptoms of Parkinson's disease. Other applications include monitoring epilepsy and/or estimating the effects of anticipated brain surgery.

A typical implantation process is long, for example an hour or two or longer and requires at least one skilled technician in addition to a physician. In the typical implantation process, an electrode lead is slowly advanced at small increments, until a signal detected at the electrode lead tip match a desired profile or until a stimulation at the electrode tip has a desired effect on the brain. This insertion typically causes some brain damage. If the electrode is incorrectly placed, it is retracted and then reinserted, typically causing additional brain damage. The accumulated damage, while not fatal, is generally undesirable and poses some degree of risk. The initial insertion into the brain typically uses an anatomical map, for example, a general map or one created by imaging the patient's brain.

European patent publication EP 1062973, the disclosure of which is incorporated herein by reference, describes an electrode body having a plurality of ring stimulation electrodes adjacent its tip and a single cell-specific sensing electrode retractably axially movable relative to its tip. Also described is the possibility that the sensing electrode acts as a stylet to bend (or prevent bending) the tip of the electrode body. Also suggested is an electrode body with several stimulation tips or asymmetric stimulation electrodes.

The following papers, the disclosures of which are incorporated herein by reference, describe techniques for measuring brain signals in vivo: “Spatiotemporal Firing Patterns in the Frontal Cortex of Behaving Monkeys”, M. Abeles, H. Bergman, E. Margalit and E. Vaadia, Journal of Neurophysiology, 1993, vol 70 No. 4, pp 1629:1638, “Microelectrode-Guided Pallidotomy: Technical Approach and its Application in Medically Intractable Parkinson's Disease”, Jerrold L. Vitek et al, Journal of Neurosurgery, 1998, “Neurons in the Globus Pallidus Do not Show Correlated Activity in the Normal Monkey, but Phase-Locked Oscillations Appear in the MPTP Model of Parkinsonism”, Asaph Nini et al, Journal of Neurophysiology, October 1995 and “Contrasting Locations of Pallidal-Receiving Neurons and Microexcitable Zones in Primate Thalamus”, Buford J A, Inase M, Anderson M E, J Neurophysiology, March 1996;75(3): 1105-16.

U.S. Pat. No. 6,011,996, the disclosure of which is incorporated herein by reference, describe electrode bodies with separate sensing and stimulating electrodes. The '996 patent also describes a micro-drive system for inserting electrodes into the brain. PCT publication WO 98/41145, the disclosure of which is incorporated herein by reference, describes a micro-drive for implanting multiple electrodes. A continuation in part of this patent has published as U.S. patent application publication US2002/0022872, the disclosure of which is incorporated herein by reference.

U.S. Pat. No. 5,458,629, the disclosure of which is incorporated herein by reference, describes an implantable ring electrode lead including multiple axially displaced rings. A stylet is suggested for use in stiffening the lead body during implantation. Use of the multiple electrodes is suggested for selective stimulation and sensing.

U.S. Pat. No. 4,809,694, the disclosure of which is incorporated herein by reference describes a threaded cranial tap used for guiding biopsy devices into the brain.

### SUMMARY OF THE INVENTION

A broad aspect of some embodiments of the invention relates to electrode designs, algorithms and/or apparatus which allow for relatively rapid and/or exact determination of electrode placement in the brain. Alternatively or additionally, less skill is required of an operator, for performing an implantation. It should be noted that in some embodiments of the invention, the use of the methods and apparatus described herein does not necessarily result in faster and/or better results.

An aspect of some embodiments of the invention relates to neural electrode leads including a plurality of electrodes that are arranged around a circumference of a lead and have a limited angle of sensing. Optionally, at least some of the thus radially localized electrodes are radially displaced from each other. Alternatively or additionally, at least some of the electrodes are axially displaced from each other. In an exemplary embodiment of the invention, the localized electrodes each comprises bi-polar electrodes, for example, an outer electrode with an associated inner electrode. In some embodiments of the invention, the electrode body is rotatable and/or axially moveable to provide multiple localized measurements of a volume. In an exemplary embodiment of the invention, the multiple measurements are used to assist in more rapidly find a desired stimulation location.

Optionally, the lead is guided using a stylet to maneuver a tip of the lead, rather than retracting the whole length of the lead. In an exemplary embodiment of the invention, manipulating the stylet will affect a plurality of sensing electrodes at a time. In an exemplary embodiment of the invention, such a stylet is used to allow side motions to reduce at least part of the time expended on trans-axial motions of the lead.

An aspect of some embodiments of the invention relates to shaped arrays of micro-electrodes, in which an array of micro-electrodes is selectively advanced relative to a main lead body and the electrode array self-deploys to be not purely perpendicular to an axis of the lead body. In an exemplary embodiment of the invention, the shape of the deployed array is that of a sphere or a surface of a sphere. Alternatively or additionally, the shape of the deployed array is an inclined plane. Alternatively or additionally, an inclined line is provided. Alternatively or additionally, a cone shape is provided. Alternatively or additionally, the array deploys as a line, having a width greater than the lead body, for example, 1.5 or twice the width. In an exemplary embodiment of the invention, the deployed array is designed to allow for some error in placement of the micro-electrodes. In combination with a method described below of using multiple measurements to localize fast, a relatively rapid positioning of stimulation and/or sensing electrodes is optionally provided.

In an exemplary embodiment of the invention, the micro-electrodes are provided as a contiguous array. Alternatively or additionally, the micro-electrodes are each advanced through a separate point in the lead body, as a discrete array.

In an exemplary embodiment of the invention, contiguous micro-electrodes are coated with a layer of sugar, or embedded in a suitable matrix, that dissolves in the brain after the electrodes are advanced, allowing the electrodes to spring to a previously trained position. Alternatively, the micro-electrodes are pre-arranged to have a desired configuration. Optionally, the configuration spreads out a small amount after the matrix dissolved. Alternatively no matrix is provided or the electrodes are rigidly coupled.

In an exemplary embodiment of the invention, the volume covered by the micro-electrodes is selected to match a placement error and/or a desired sensing volume, for example, a length, width and/or depth of 1, 2, 5, 10, 15, 20 mm, or any smaller, intermediate or larger dimension.

In an exemplary embodiment of the invention, the electrodes are selected to advance a distance sufficient to nullify any inference caused by the physical interaction of the lead body with brain cells (e.g., 100-150 microns). The advancing distance may be, for example, 200-300 microns. This may nullify disruption caused by such physical interaction. Optionally, the electrodes are advanced significantly more than this distance, for example, 15 mm. In an exemplary embodiment of the invention, an oblique array is provided and 15 mm indicates the distance of the furthest part of the array.

In an exemplary embodiment of the invention, the micro-electrodes are provided using a guiding tube. Optionally, the electrodes and/or the tube remain implanted in the body. The tube optionally includes thereon one or more stimulation and/or sensing electrodes. Alternatively or additionally, the micro-electrodes are used as stimulation electrodes, with, optionally, some of the micro-electrodes being shorted together.

An aspect of some embodiments of the invention relates to a method of determining a target area for an electrode, in which measurements of a brain area are simultaneously taken from locations straddling the area. The measurements are analyzed to determine the position of the electrodes relative to the area and/or a specific location therein. Optionally, these measurements are analyzed for correlation.

In an exemplary embodiment of the invention, multiple measurements are taken without moving the lead, instead of taking multiple measurements with interspersed micro-movements of the lead. Thus, exact axial positioning of the lead is less important and/or less time is expended on exact positioning of the lead.

In an exemplary embodiment of the invention, making multiple measurements for each movement of the lead accelerates the positioning process, for example to be 1, 3, 5, 10, 30 minutes or less from insertion of the lead to position determination. Alternatively or additionally, the positioning process is made more exact. Alternatively or additionally, if the positioning is for later stimulation, the stimulation electrode is positioned or activated according to the sensing.

An aspect of some embodiments of the invention relates to locating a brain location using one or more composite sensing steps, in each of which steps electrical activity in a plurality of brain areas are sensed. In an exemplary embodiment of the invention, a plurality of measurements are analyzed to determine an exact or more exact (than in a previous estimation) location. Then, a lead used for the measurements is optionally moved to provide another set of measurements. In an exemplary embodiment of the invention, when the lead is inserted, it is inserted in one step to a location that is (e.g., at least one of its electrodes) definitely past the searched-for location. The lead is optionally a multi-electrode lead having electrodes straddling the searched-for location. Alternatively or additionally, the lead may be moved in large jumps, optionally as long as the straddled areas have some overlap, thus possibly obviating or reducing the need for slow micro-drive advancement.

In an exemplary embodiment of the invention, the distance between electrodes on the lead is selected to ensure straddling of the searched-for location, in view of expected errors, for example, an imaging error and a brain-shift error. Alternatively or additionally, the spatial density of electrodes is selected to ensure that at least one electrode will be near enough to the searched-for location, for effective stimulation and/or for ensuring that the signals at the location will be detected.

In an exemplary embodiment of the invention, an automatic recognition algorithm is used to detect a match between the measurements and/or correlation and desired and/or expected properties of the target area. The automatic detection may be used as feedback to control advancing and/or retraction of the electrode lead and/or determining that a desired location was found. Alternatively or additionally, the automatic detecting is used as input to decide if to provide stimulation at the location and/or what stimulation to apply. In an exemplary embodiment of the invention, if a certain expected correlation between activity of cells is not found, it is assumed that the patient is sleeping and/or that no abnormal activity exists, so no stimulation need be applied.

An aspect of some embodiments of the invention relates to determining a functional position in a brain based on the relationship between electrical activity at a plurality of electrodes of a multi-electrode lead. In an exemplary embodiment of the invention, the position is determined automatically. Alternatively or additionally, these methods are used to automatically detect motion of the lead. Optionally, one or more electrodes in a lead are set aside for detecting position and/or movement.

In an exemplary embodiment of the invention, the position is determined by detecting a correlation between signals sensed at a plurality of electrodes and/or positions. Alternatively or additionally, the position is determined by detecting a match between an expected spatial distribution of the signals and an actual spatial distribution of the signals. Alternatively or additionally, the position is determined by detecting a particular response at one electrode to a stimulation at a different electrode on the same lead.

An aspect of some embodiments of the invention relates to a method of setting stimulation parameters in which correlation between micro-electrode recording of single cells are used. In an exemplary embodiment of the invention, recordings from multiple cells are analyzed to determine activity chains inter-linking the cells. Stimulation parameters are determined and tested. The effect of a stimulation parameter is optionally assessed based on its effect on the activity chain. Alternatively or additionally, a plurality of electrodes are electrified at a same time. Alternatively or additionally, the effects of stimulation are determined and/or correlated with measurements of the patient's body response, e.g., tremors and rigidity. In an exemplary embodiment of the invention, a range of electrification locations and/or electrification parameters are tested automatically. In an example of motion disorder, the effect tested for may be an effect on oscillatory behavior of the cells.

An aspect of some embodiments of the invention relates to a cap for a cranial tap and a method of using the cap. In an exemplary embodiment of the invention, an electrode tap is provided with a cap. In use, a tap is installed and capped and then the patient is imaged. The electrode driver is then mounted (e.g., using a screw or other mechanical attachment method) on the cap and registered to the image. Using a capped tap allows the imaging to be performed in a separate room from the tap installation and prevents inference of the drive mechanism with the imaging. In an exemplary embodiment of the invention, the tap includes a rigid connector to which the drive can be attached in a known manner.

There is thus provided in accordance with an exemplary embodiment of the invention, a multi-electrode lead for neural applications in the brain, comprising:

an elongate body having a tip and an axis; and

a plurality of electrodes arranged at said tip, each of said electrodes having a limited angular sensitivity relative to said axis. Optionally, said electrodes are radially separated. Alternatively or additionally, said electrodes are axially separated. Alternatively or additionally, said electrodes are single cell sensing electrodes. Optionally, said electrodes are selectively extendible.

There is also provided in accordance with an exemplary embodiment of the invention, a multi-electrode lead, comprising:

a delivery tube adapted to be inserted into a brain and having an axis; and

a plurality of micro-electrodes which are provided through said tube, said micro-electrodes having sensing areas which define a surface, wherein said surface is not a plane perpendicular to said axis. Optionally, said surface is planar and inclined to said axis. Alternatively, said surface is curved.

In an exemplary embodiment of the invention, said electrode tips define a sensing volume which is bounded by said surface on at least one side thereof.

There is also provided in accordance with an exemplary embodiment of the invention, a multi-electrode lead, comprising:

a delivery tube adapted to be inserted into a brain and having an axis; and

a plurality of micro-electrodes held together by a water soluble material and being pre-stressed to deploy by moving apart when said material dissolves. Optionally, said electrodes move apart at least 200 micro meters, from each other, when they deploy.

There is also provided in accordance with an exemplary embodiment of the invention, a multi-electrode delivery system, comprising:

a lead body having an axis and defining at least one stimulation electrode; and

a plurality of micro-electrodes, wherein said micro-electrodes are adapted to be delivered along said axis. Optionally, said micro-electrodes are provided through a channel of said lead. Alternatively, said micro-electrodes are provided through a guide tube that encloses said lead.

In an exemplary embodiment of the invention, at least some of said micro-electrodes are held together by a water soluble material and are pre-stressed to deploy by moving apart when said material dissolves.

In an exemplary embodiment of the invention, said at least one stimulation electrode comprises a plurality of axially spaced stimulation electrodes.

There is also provided in accordance with an exemplary embodiment of the invention, apparatus for locating a location in the brain, comprising:

means for detecting electrical signals from a plurality of locations in the brain;

means for detecting correlation between the detected signals; and

computing means for determining said location based on said correlation. Optionally, said means for detecting comprises means for simultaneously detecting. Alternatively or additionally, said means for detecting comprises a plurality of implanted spaced apart electrodes adapted to ensure straddling of said location.

There is also provided in accordance with an exemplary embodiment of the invention, a method of implanting an electrode in a brain, comprising:

advancing a multi-electrode lead past an estimated location of interest in the brain;

sensing signals from electrodes of said lead; and

analyzing said signals to generate a more exact estimate of said location. Optionally, the method comprises selectively stimulating at said more exact estimate of location, to effect a treatment of a patient.

There is also provided in accordance with an exemplary embodiment of the invention, a method of locating a position of a functional location in a brain, comprising:

detecting signals from a plurality of locations in a brain, which locations have a known physical positional relationship; and

correlating a behavior of said signals to determine a position of a specific functional location of the brain.

Optionally, the method comprises:

assuming a function of said brain at a position; and

using said correlation to verify said function.

In an exemplary embodiment of the invention, said plurality of locations comprises functional locations. Alternatively or additionally, said plurality of locations comprises physical locations.

In an exemplary embodiment of the invention, correlating comprises comparing to a database of functional signals. Alternatively or additionally, correlating comprises matching a spatial pattern of said signals to an expected pattern. Alternatively or additionally, correlating comprises matching between signals of different locations.

In an exemplary embodiment of the invention, detecting comprises detecting simultaneously. Alternatively or additionally, correlating comprises detecting a response of a signal at at least one location to stimulation at a second location.

In an exemplary embodiment of the invention, said signals are single cell signals and the method comprises setting parameters for stimulation of the brain responsive to said detected signals.

There is also provided in accordance with an exemplary embodiment of the invention, a cranial tap, comprising:

a body having an aperture therein, wherein said body is adapted to be attached to a hole in a skull and adapted to have mounted thereon a guide for an intra-cranial electrode lead that passes said aperture; and

a cap adapted to seal said aperture of said body after insertion of said body into said hole.

### BRIEF DESCRIPTION OF THE FIGURES

FIG. 1 is a schematic showing of a body portion including a brain and having an indication of a location in which to implant an electrode.

FIG. 2 is a schematic section view of a micro-drive tap for advancing electrodes in accordance with an exemplary embodiment of the invention;

FIGS. 3A and 3B are flowcharts of a process of positioning an electrode lead at a target location, in accordance with an exemplary embodiment of the invention;

FIGS. 4A-4E illustrate various embodiments of tips of multi-electrode leads, in accordance with exemplary embodiments of the invention;

FIGS. 4F-4K illustrate micro-electrode array configurations, in accordance with an exemplary embodiment of the invention;

FIGS. 5A and 5B illustrate lead tips including a stylet, in accordance with exemplary embodiments of the invention;

FIG. 6 is a flowchart of a method of determining stimulation parameters, in accordance with an exemplary embodiment of the invention; and

FIG. 7 is a flowchart of a method of abnormal activity detection, in accordance with an exemplary embodiment of the invention.

### DETAILED DESCRIPTION OF EXEMPLARY EMBODIMENTS

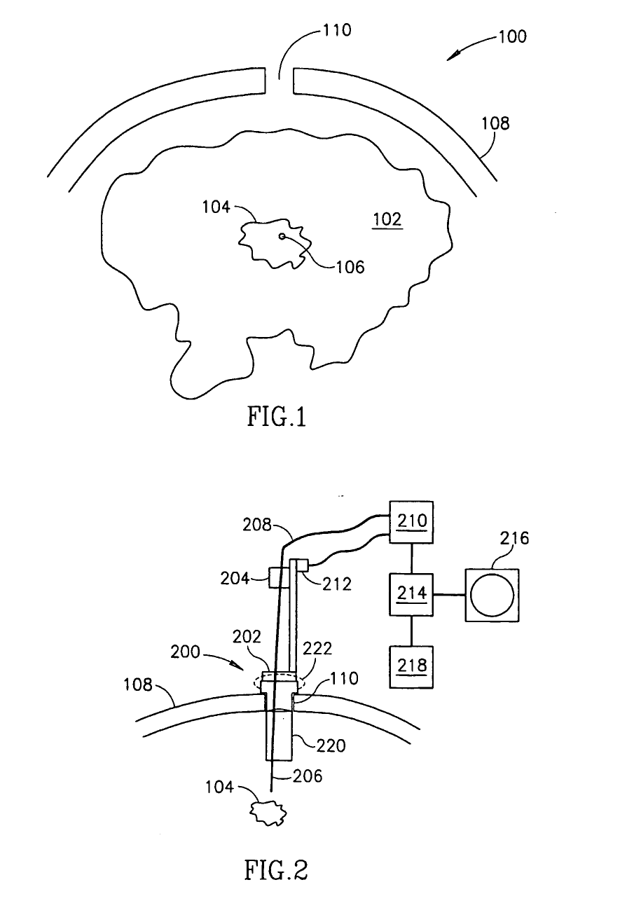

FIG. 1 is a schematic showing of a body portion 100 including a brain 102. An electrode is to be positioned at a location 106 in the brain, for example, in order to effect treatment or take measurements. In an exemplary procedure, an electrode (not shown) is advanced through an opening 110 (e.g., a bore hole) in a skull 108, to a brain region 104 that includes location 106. Typically, brain region 104 is detected on medical images of the patient. This detection is used to plan the location of opening 110 and the trajectory of the electrode. Typically, however, the exact position of location 106 cannot be determined from the images, being functional in nature, or even if it can be determined, is difficult to aim for. Thus, a search procedure is used to find location 106.

FIG. 2 is a schematic sectional view of a micro-drive tap 200 for advancing electrode leads in accordance with an exemplary embodiment of the invention.

In the embodiment shown tap 200 is attached directly to the skull, for example, using adhesive, screws or being screwed into opening 110. Alternatively, tap 200 may be mounted on a stereo-tactic frame. An optional aiming mechanism 202 is used to aim the trajectory of the electrode. In an exemplary embodiment of the invention, the patient's head is imaged after the tap (or at least a base of the tap) is attached to the head, to assist in registering the tap coordinates to the brain imaging coordinates.

A micro-drive 204 is used to incrementally advance and retract an electrode lead 206 towards region 104. While substantially any micro-drive may be used for this task, it should be noted that in some embodiments of the invention, relatively gross-positioning of the lead is allowed, so possibly, no micro-drive is required. Optionally, a graduated scale is provided on the electrode insertion mechanism, for assisting approximate manual insertion.

Optionally, a second microdrive (not shown) is used for non-axial (e.g., angular and/or trans-axial) movement of lead 206. Stimulation signals to the lead and/or sensing from the lead is conveyed by a plurality of wires 208 that connect lead 206 to a controller 210. Alternatively, an on-tap or on-lead controller (not shown) is used. An optional preamplifier 212 (not shown connected to lead 206) is provided to amplify sensed signals. Controller 210 and/or a different controller may be used to control microdrive 204.

In an exemplary embodiment of the invention, a complete tapping system includes a computer 214 with a display 216 and a database 218. Computer 214 can serve to instruct controller 210 on settings and moving lead 206 and/or be used for data storage, for data analysis and/or for user interface. Alternatively or additionally, display 216 is used to display system status and/or sensing results. Alternatively or additionally, database 218 is used to store images and/or signals, for example for uses as described below.

Optionally, lead 206 is provided through a guide tube 220 that has a wider inner diameter than the lead and is shorter therefrom. In an exemplary embodiment of the invention, lead 206 is not axially positioned in tube 220 and small trans-axial and/or angular motion of lead 206 is achieved by changing its position and/or orientation in tube 220. In one example, tube 220 includes one or more inserts, each with one or more off-axis apertures for receiving lead 206. Rotating and/or axially moving the inserts changes the orientation and/or trans-axial position of lead 206.

Optionally, tap 200 has a matching cap 222 (shown as a dotted line). Between procedures, leads 206 (or their external connections), microdrive 204 and/or aiming mechanism 202 are removed and tap 200 is capped. One possible use of capping is allowing the insertion of the tap and calibration of its position to be performed in a radiology OR (operating room) and the electrode implantation be performed in a different OR or EP (electro-physiology) laboratory, without danger of infection.

Alternatively or additionally, tap 200 and/or cap 222 are MRI and/or CT compatible. In an exemplary embodiment of the invention, the entire procedure described below is performed while imaging using an intra-operative MRI imager. Optionally, electrical measurements from lead 206 are correlated with functional MRI images of the brain.

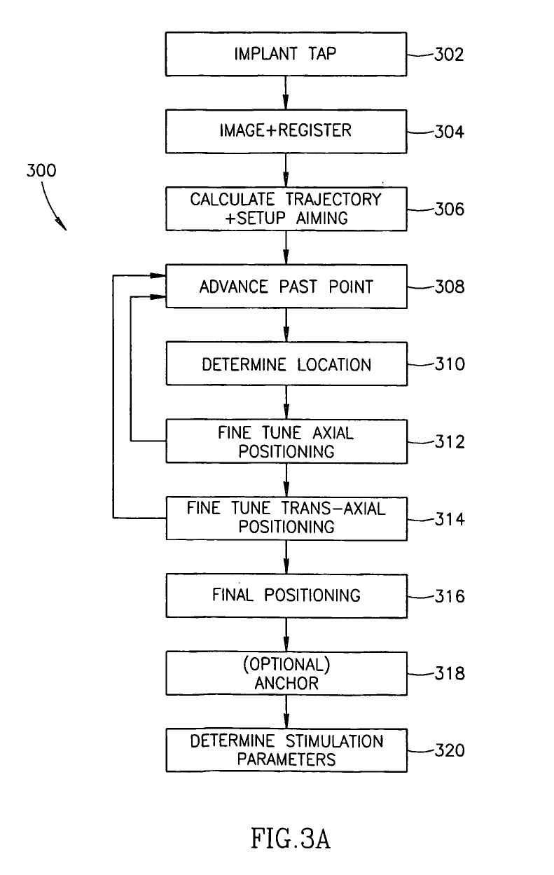

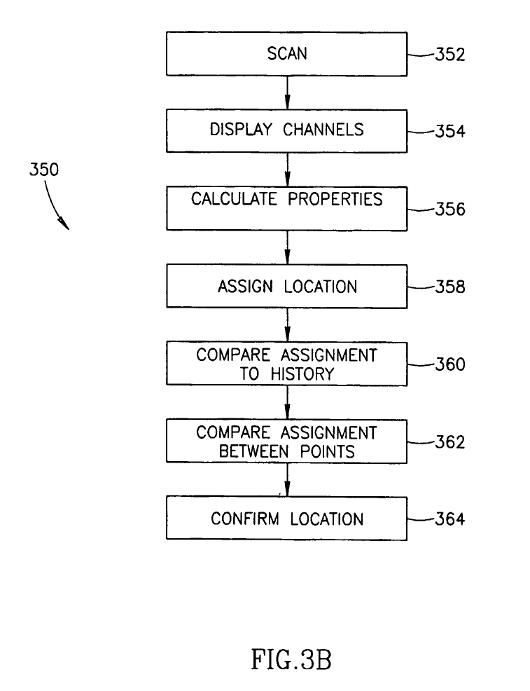

FIGS. 3A and 3B are flowcharts 300 and 350 respectively of a process of positioning electrode 206 at location 104, in accordance with an exemplary embodiment of the invention.

At 302 tap 200 is implanted, using, for example, any method as known in the art, for example inserting a tap manufactured by IGN (Image Guided Neurologics) or by FHC (Frederick Haer Corporation) into a bore hole drilled/cut in the skull.

At 304, the patient is imaged and the tap coordinates are registered to the image coordinates, in 2D and/or 3D. The probable position of location 106 is determined on the image. Typically, the gross position and/or alignment of tap 200 is determined based on a gross estimate of location 106.

At 306, a trajectory from tap 200 to location 106 is calculated, possibly taking into account any possible movement of brain 102. Aiming mechanism 202 is set up to accommodate the trajectory and controller 210 or computer 214 (depending on the system configuration) are set up with an estimated distance to location 106 along the trajectory. If a curved lead or a lead with a bendable tip are used, the trajectory design optionally takes this into account, for example, to bypass sensitive parts of the brain.

In an alternative embodiment of the invention, the lead, aiming mechanism and optional micro-drive are mounted on a stereo-tactic frame, as known in the art, and the lead is aimed using the frame. Typically, a frame base and/or a plurality of fiduciary screws are attached to the patient before imaging (e.g., for registration) and after imaging an arc (on which the micro-drive is later mounted) is attached on the base.

In an exemplary embodiment of the invention, a user inputs the target into the system, so that the system can determine which measurements should be detected while the electrode is being inserted and/or after it is inserted. Optionally, one or more measurements are made during insertion to ensure that the expected trajectory is being followed. “Standard” data for comparing to the actual measurements may be obtained, for example, from the same or different patients or from multiple patients. Alternatively, the “standard” data may be simulation results.

At 308, lead 206 is advanced to and optionally past the estimated position of location 106. This allows measurements to be taken in a manner that straddles location 106. If multiple sensing electrodes are provided on lead 206, a single, advancing step may be performed. If only a single electrode is provided, or the electrodes are too close together to allow straddling, measurements are taken before reaching, at and past location 106.

At 310, the position of location 106 is determined, for example as will be described below with reference to FIG. 3B.

At 312 and 314, the axial (e.g., along the trajectory) and/or trans-axial (e.g., perpendicular to the trajectory) positioning of lead 206 are optionally adjusted, for example, to iteratively determine the position of location 106 in a more exact manner. Axial positioning may include, for example, axial motion of lead 206, which may be automatic, for example, within bounds defined to controller 210. Trans-axial positioning may include, for example, bending a tip of lead 206 and/or retracting and reinserting lead 206, or rotating lead 206 (if it has directionally positioned electrodes, for example).

At 316, lead 206 is given a final positioning, if necessary, for example, based on determination at 310-314. In some cases, lead 206 is replaced with a different lead, for example for specific applications. However, it is noted that at least some of the electrodes described herein may be used both for locating and, later, for stimulation, so no replacement is required for those electrodes.

Optionally, at 318, an anchor is provided to anchor lead 206 in location. In an exemplary embodiment of the invention, the anchor is provided at the tip of lead 206. Alternatively, the lead is anchored by pouring a bio-compatible cement into opening 110, to seal the bore hole and fix the lead. Alternatively, a cap is used to seal the bore hole and fix the lead in place. Exemplary such caps are manufactured by Medtronic and by IGN.

In an exemplary embodiment of the invention, the distal end of lead 206 is attached to a programmable stimulator. The stimulator may be on the skull. Alternatively, the lead is conveyed under the skin to the chest, where a simulator is implanted.

At 320, stimulation parameters are optionally determined, for example as described below. The parameters may be determined after the surgery is completed, as well.

FIG. 3B shows a detail of 310 (FIG. 3A), as flowchart 350, in accordance with an exemplary embodiment of the invention.

At 352, all the sensing channels of electrodes of lead 206 (e.g., there may be more than one) are scanned for signs of neural activity, as some of the electrodes may not be adjacent an active cell or any cell at all.

At 354, all the channels that have activity are optionally selected and, optionally, displayed. In an automatic systems, these channels may not be displayed.

At 356, one or more properties are calculated for each channel. For example, these properties may include one or more of:

(a) spike characteristics, such as spike shape, spike amplitude, average firing rate and/or firing pattern;

(b) local field potentials (LFP) characteristics, such as amplitude and spectrum;

(c) correlation with previous recordings of the same or other electrodes in this or other brains and/or correlation with current recordings;

(d) reaction to stimulation;

(e) reaction of one area to a stimulation of another area, for example, inhibition of Palidal activity in response to Striatum stimulation; and/or

(f) depth of electrode, which generally indicates expected spike characteristics and other details about local activity.

At 358, a location is assigned to each electrode, which associates an apparent functional behavior of the electrode with an apparent (relative or absolute) anatomical position. Alternatively a distance of the electrode from target location 106 is estimated. This location may be determined, for example, on a match between the characteristics of the channel and the previously stored values. In an exemplary embodiment of the invention, the location is overlaid on an anatomical map. Optionally, a user can override the automatically assigned location. Alternatively, some or all the locations are provided manually to begin with. An automatic method may be used to provide feedback on the quality of the manual decision.

At 360, the assigned location is optionally compared to a historic location of the electrode, in this and/or a previous procedure, possibly taking into account axial and/or trans-axial movement.

At 362, the assigned locations of several electrodes are optionally compared to each other and/or to an expected spatial order of the electrode properties. If 360 and/or 362 do not succeed, for example, giving matches below a threshold; sensing, calculation and/or evaluation are optionally repeated. In some cases, a majority vote is used to assign the location. Alternatively or additionally, other methods are used, for example center of gravity methods. If no match is found, low probability matches may be shown to a user to select between or for the user to decide on a new trajectory.

At 364, the assigned locations are optionally confirmed, for example, by stimulation and sensing the response (of the brain and or the body) to stimulation.

In an exemplary embodiment of the invention, some of the above steps use a database of neural traces and/or properties, for example from the same or from different patients. Different traces are optionally associated with relative spatial locations in area 104 and/or in brain 102 as a whole (e.g., absolute and/or relative positions).

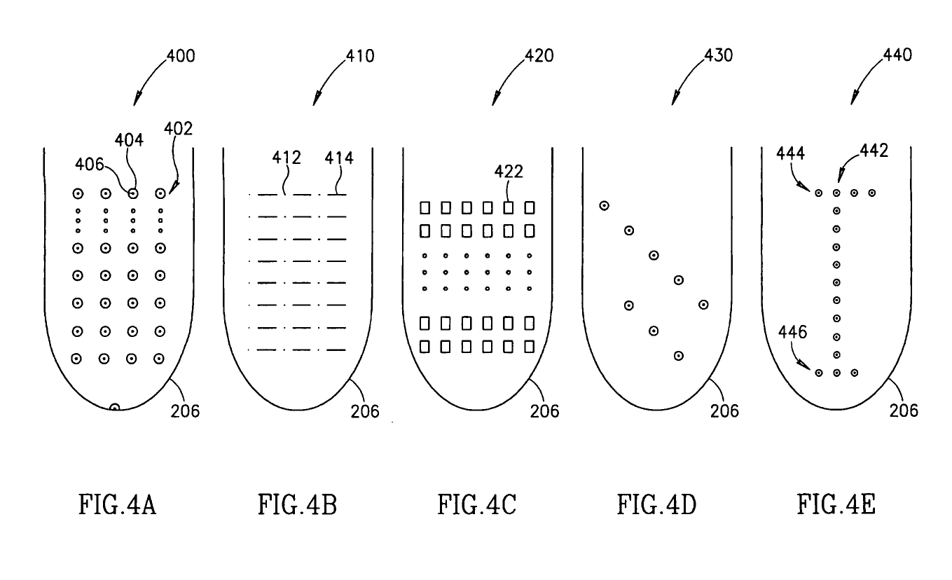

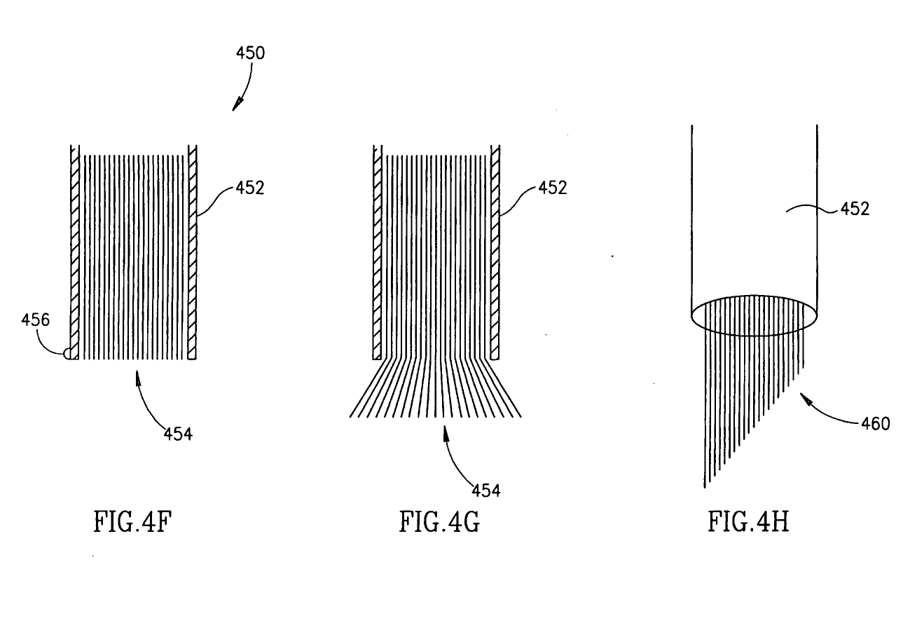

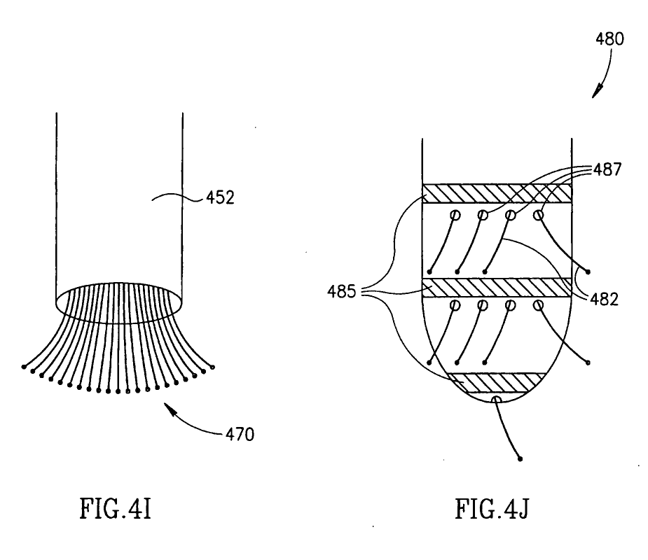

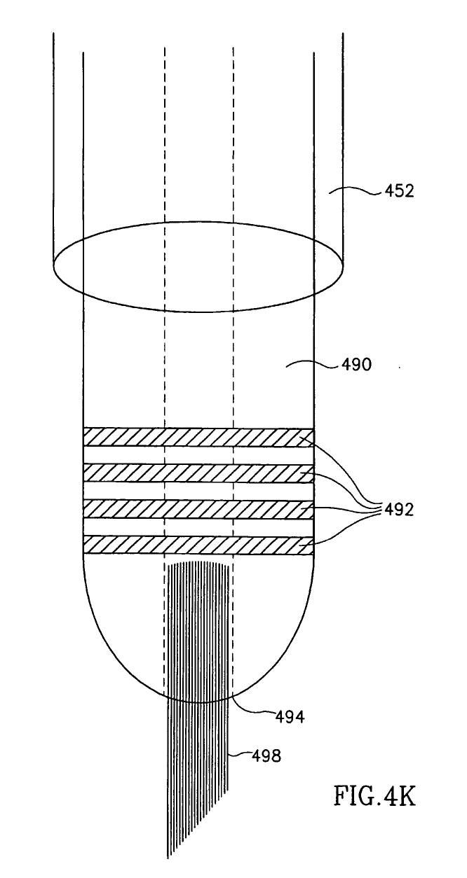

FIGS. 4A-4E illustrate various embodiments of tips of multi-electrode leads, in accordance with exemplary embodiments of the invention. Other multiple-electrodes arrangements are also considered to be within the scope of various embodiments of the invention.

FIG. 4A shows a tip 400, having a plurality or rows of electrodes 402, each row including a plurality of electrodes. The electrodes optionally reach the tip of lead 206. The rows may have the same number of electrodes or they may, for example, taper. Alternatively or additionally, the distance between rows may be constant or vary. An electrode 402 comprises, for example an outer ring electrode 404 and an inner point electrode 406, which may be operated, for example, as a bipolar electrode. The electrodes in tip 400 may all be of a same kind or they may be of varying types, for example, as described below. In this and other electrode designs, smaller electrodes (e.g., of sizes of 25 microns) are optionally used for stimulation and/or sensing of micro-volumes (e.g. single or small numbers of cells) and larger electrodes are used for stimulation and/or sensing of macro-volumes (e.g., multiple cells and general activity).

FIG. 4B shows a tip design 410, in which two type of electrodes are used, point electrodes 412 and line electrodes 414.

FIG. 4C shows a tip design 420, having a plurality of point or small plate element electrodes 422 of a same kind.

FIG. 4D shows a tip design 430 in which the electrodes are arranged as a spiral, for example around the axis of the lead.

FIG. 4E shows a tip design 440, having two rings of point electrodes 444 and 446. These rings can straddle location 106 and provide an indication of whether location 106 is between them or not. They can be rings having multiple electrodes as shown or be simple unipolar or bipolar rings, for example. Alternatively or additionally, a column 442 is provided. By rotating lead 206, the direction of location 106 can be ascertained. Alternatively or additionally, a more exact axial location is determined by reading from the electrodes between the rings. In an exemplary embodiment of the invention, this design allows fewer actually addressable electrodes to be used than if a complete, dense, matrix of addressable electrodes were provided on the lead.

In an exemplary embodiment of the invention, a plurality of electrodes can be shorted together using, for example, a switch array outside of lead 206 to sense signals at a larger area. Alternatively or additionally, a common ground electrode (not shown) is used. Other, more complex effects may be provided as well, for example as used in multi-element electrode arrays in the heart.

In an exemplary embodiment of the invention, a plurality of electrodes are shorted together to define various directional active channels of lead 206. Optionally, the physical electrodes are grouped to define logical electrodes that have various effective depths of detection. This may allow to detect a distance of location 106 from lead 206.

Alternatively or additionally, electrodes are shorted together to define large electrodes suitable for stimulation.

It should be noted that by using multi-element electrodes, the electrification field and/or sensing field of a lead may be fine-tuned even after anchoring, for example, by changing switch addressing to define different active electrodes. Thus, physical fine tuning and positioning, and their associated time, labor and dangers, can be avoided in some embodiments of the invention.

In an exemplary embodiment of the invention, 1, 2, 4, 6, 8 or a smaller, greater or intermediate number of electrodes are provided on the circumference of a lead. The electrodes may cover a large sector or a small sector, for example, 1°, 10°, 25° or 45°, or any smaller greater or intermediate angular size. The number of electrodes and/or their angular extent may vary along the lead and/or around the circumference. The number of rows may be, for example, 2, 5, 10, 20 or any intermediate or greater number of rows. The rows may be equidistant or not, for example, having a greater density near the ends of the electrode area and/or at its center. The active electrode area may be, for example, 0.5, 1, 2, 4 mm or any smaller greater or intermediate length. Thus, a lead may include, for example, 5, 10, 20, 40 or any smaller larger or intermediate number of electrodes.

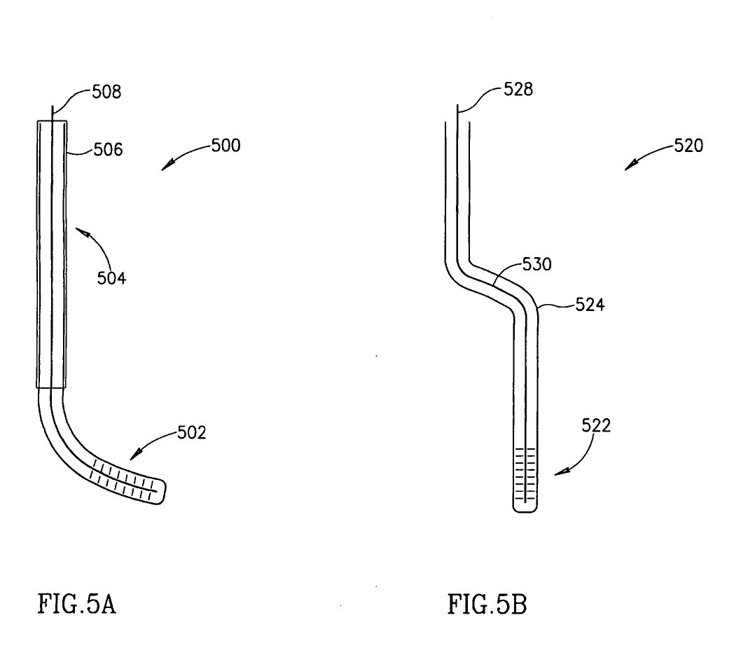

In an exemplary embodiment of the invention, lead 206 has a diameter of 0.8-1.3 mm, however, the lead may have a non-circular cross-section, for example, elliptical, flat or twisted. The present invention is not limited to these particular sizes and shapes, which are for illustrating exemplary embodiments. The lead is desirably formed of bio-compatible material (e.g., plastics) as known in the art having a plurality of wires embedded therein. Pads for the electrodes may be formed as part of the wires, or be separately attached. In one example, the pads are gold pads embedded on the lead surface. Alternatively, in some embodiments, other lead structures as known in the art may be used. Optionally, a lumen is provided for a stylet, for example as described below (e.g., FIGS. 5A, 5B). Alternatively, the stylet is an external over-tube, provided over lead 206. In other applications, for example, for the spine, other lead sizes may be used.

FIGS. 4F-4K illustrate micro-electrode array configurations, in accordance with an exemplary embodiment of the invention.

FIG. 4F shows an electrode assembly 450, including a lead body 452 enclosing a micro-electrode array 454. Each electrode may be, for example, between 15 and 100 microns in diameter. The wires are shown at the tip of lead body 452, so that they can measure some signals forward of the lead. Alternatively or additionally, lead 452 includes one or more sensing electrodes 456 used for position determination. Alternatively or additionally, the micro-electrodes are advanced out of body 452 for such measurements and then optionally retracted. Alternatively or additionally, a single micro electrode is so advanced. In an exemplary embodiment of the invention, 5, 10, 20, 30, 40 or any smaller, intermediate or larger number of micro-electrodes are provided.

Once the micro-electrodes are advanced, in an exemplary embodiment of the invention, it is desirable that they separate. However, this may interfere with other movements of the micro-electrodes. In an exemplary embodiment of the invention, the micro-electrodes are attached to each other, for example using a bio-absorbable matrix, such as sucrose. After a time in the brain, this sucrose dissolves and the wires can separate. Optionally, the wires are pre-stressed to have a particular separation. It is noted that the time at which the wires separate may be selected to be long enough so that one or more sessions of advancing and retracting the electrodes as a whole, for positioning purposes, may be practiced. Optionally, the tip of the electrodes are separated, to all some distance between the electrodes. Alternatively, for example as described below, the micro-electrode array is tapered. In an exemplary embodiment of the invention, the matrix material is selected to dissolve after 1, 10, 30, 100, 200 or any smaller, intermediate or larger number of seconds.

FIG. 4G shows micro-electrode array 454, after it spread out. In this example, spread array 454 is circular and planar.

FIG. 4H shows a linear tapered array 460, in which, for example, the micro-electrodes are each displayed axially by 0.5 mm from adjacent electrodes. Other distances and non-uniform distances may be used as well. In an exemplary embodiment of the invention, the distances are selected to straddle the range of axial positions expected due to error considerations and/or area of interest of activity considerations, for example, 15 mm. In an exemplary embodiment of the invention, the micro-electrodes are distanced from each other and from the lead body at least 50-150 microns or any other distance in which a negative effect on signal sensing and/or cellular activity is precipitated by the lead body.

FIG. 4I shows a sphere surface micro-electrode array 470. In the example shown, the surface is that of part of a sphere, shown for simplicity as an arc. Alternatively or additionally, a complete sphere may be provided. In an exemplary embodiment of the invention, the sphere is selected to be a 10 mm diameter, to compensate for an expected 4 mm positioning error. While a surface is shown, in some embodiments of the invention, the electrode tips define a volume, for example half a sphere.

In an alternative exemplary embodiment of the invention, the electrodes are not all stuck together. Instead, each electrode, or group of electrodes has a defined exit hole. For example, any of the electrode patterns shown in FIGS. 4A-4E may be used. FIG. 4J shows an example of such an electrode array 480, in which a plurality of micro-electrodes 482 are extendible from a plurality of apertures 484. Optionally, the electrodes are all extended together. Alternatively or additionally, some or all of the electrodes are individually controllable, for example manually or using a micro-drive. Optionally, the use of the micro-electrodes takes into account the flexibility of the micro-electrodes. For example, if more precise positioning is required, the electrodes are not extended to far. Conversely, if a larger volume is required, the electrodes may be advanced further. In some embodiments of the invention, the micro-electrodes are made of stiffer material. In others, they are more flexible. Optionally, each micro-electrode is provided with an individual sheath or lumen. While in an exemplary embodiment of the invention, the micro-electrodes are cell electrodes (e.g., unipolar or bipolar), alternatively, they are small area electrodes, possibly sensing electric fields along a length of the electrode.

In an exemplary embodiment of the invention, if one of the micro electrodes is found to be in an exactly desired stimulation location, the lead body may be removed and the single micro-electrode used for stimulation or a stimulation electrode guided over it (e.g., before or after the lead body is retracted). Thus, in some embodiments of the invention, a more exact stimulation electrode location is provided without trans-axial motion of the lead (e.g., retraction and then reinsertion).

In an exemplary embodiment of the invention, one or more optional stimulation electrodes 485 are provided, for example for selective stimulation, theses electrodes may have a limited radial extent, for example, using the features of FIGS. 4A-4E. Alternatively or additionally, the micro-electrodes are used for stimulation.

FIG. 4K shows an alternative embodiment in which a plurality of micro-electrodes in an array 498 are combined with a stimulation array electrode 490. In the example shown, stimulation array electrode 490 comprises a plurality of electrodes 492 and is provided through a guide tube 452. Micro-electrode array 498 is used, as a selectably advancable and retractable array for example to sense a correct position, for example using an oblique linear array as shown. Other array geometries may be used instead. In an exemplary embodiment of the invention, when the sensing electrodes sense a correct location, the stimulation electrode is advanced to cover them and they and the tube are removed, leaving only the stimulation electrode, possibly implanted for long term. In an alternative embodiment, stimulation electrode 490 is inserted into the body only after the correct position is sensed. Optionally, electrode array 498 travels in tube 452 side by side (optionally in a separate lumen) with stimulation electrode 490, instead of riding over the micro-electrodes using a central channel 494 as shown or a side channel. Alternatively or additionally, more than one stimulation electrode 490 is inserted.

FIGS. 5A and 5B illustrate lead tips including a stylet, in accordance with exemplary embodiments of the invention. Many types of stylets are known in the art of catheters. However, unlike blood vessels, in neural tissue, it is generally desirable that the lead not move trans-axially along its length when the stylet is moved, for fear of damage to brain tissue.

FIG. 5A shows a lead 500 including a tip 502 with schematically shown multiple electrodes. A body part 504 of lead 500 comprises a stiff wall 506 that is stiffer than a stylet 508. However, once the stylet enters tip 502, it can bend the tip. All or only some of tip 502 may be bent by stylet 508.

FIG. 5B shows a lead 520 having a stylet 528 that bends lead 520 in a section of a body 524 thereof that is distanced from a tip 522 thereof. In an exemplary embodiment of the invention, lead 520 is retracted (e.g., back to a bend 530 of the stylet) and the stylet advanced such that bend 530 does not affect important neural tissue or is contained by an outer stiffener tube (e.g., tube 220). Then, lead 520 is advanced on stylet 528, in a new trajectory determined by bend 530.

In general, the stylet may remain enclosed by the lead. Alternatively, the stylet itself may serve as an electrode.

In an exemplary embodiment of the invention, the type of electrode, stylet and/or micro-electrode array used is dependent of the error characteristics of the target to be located for example, for a wide but flat target, a tapered (e.g., inclined line) micro-electrode is less likely to miss. For a deep but narrow (e.g., from the direction of the approach chosen) target, a wider spreading micro-electrode array may be selected. In addition, depending on the relative errors of trajectory and depth, different shapes of micro-electrode volumes may be selected.

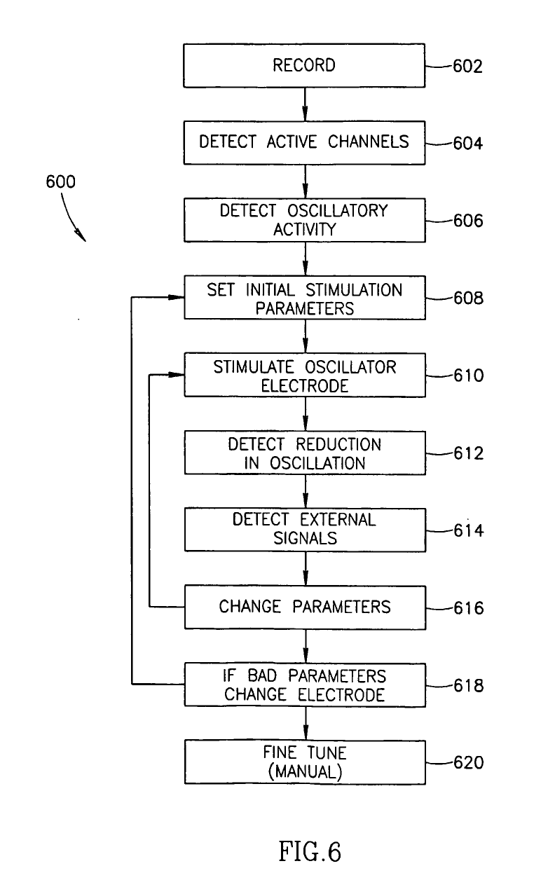

FIG. 6 is a flowchart 600 of a method of determining stimulation parameters, in accordance with an exemplary embodiment of the invention. Optionally, this method is applied a few days after lead implantation (e.g., to allow any trauma to heal). The determination may be used, for example, to determine optimal stimulation parameters for a certain therapeutic effect, for example, treating symptoms of Parkinson's disease. In an exemplary embodiment of the invention, the method is useful for multi-electrode leads in which there is a large range of possibilities to check and/or freedom to correct inexact placement of electrodes. Alternatively or additionally, the method utilizes an availability of multiple and possibly associated stimulation and sensing volumes. It should be noted that the implanted lead may be used for measurements in a therapeutic stimulation (and/or data gathering) for other applications, such as epilepsy, pain, mental disorders and/or other functional diseases and application.

At 602, recording from a plurality of channels is initiated.

At 604, electrodes that exhibit neural activity are identified. In one example, such activity is identified based on signal characteristics and/or using well known methods such as template matching, window discrimination and level crossing. The MSD “Multi-spike Detector” package is manufactured by Alpha Omega of Nazareth, Israel and includes these features. Another example analysis package is AlphaSort, by the same manufacturer, which detects and sorts unit activity according to principle components analysis.

At 606, oscillatory behavior is identified, for example using automatic methods well known in the art.

At 608, initial stimulation parameters for at least one of the oscillatory electrodes is set. Such parameters may include, for example, frequency, amplitude, envelope, charge balancing, trains of spike parameters and/or shape of stimulated volume (e.g., number and/or type of participating electrodes). For example, a frequency of about 300 Hz and a voltage of a few volts may be used.

At 610, test stimulation using the initial stimulation parameters is applied at or near one or more oscillatory electrode. In an exemplary embodiment of the invention, the initial values are selected based on a database of parameter values known to be useful for the treated disorder. The electrodes are selected, for example, based on their proximity to the oscillatory behavior. Alternatively, some or all the electrodes are tested, for example electrodes are selected all along the lead to see which electrodes might have a beneficial effect.

At 612, a reduction in oscillatory behavior is checked for. Possibly, the detection uses the same electrodes as used for stimulation and/or it uses different electrodes. The sensing may be of the stimulated volume or of a nearby volume. In an exemplary embodiment of the invention, all the channels near location 106 are monitored for oscillatory behavior and the electrodes are tested one by one (or in groups) to detect which electrode has the desired effect, optionally, with a smallest side effect. Optionally, a score is defined for the effect of the electrode, size of current and/or magnitude of side effects, to assist in selecting the final stimulation parameters.

Optionally, or for only some of the test parameter settings, a determination of external symptoms is made, for example, rigidity, tremor and bradykinisia (614). These symptoms may be measured manually or automatically. For example, acceleration sensors, questioning the patient, tremor detecting gloves and/or EMG may all be used to assess the effect of stimulation and lack thereof in motion disorders.

At 616, the parameters are changed, e.g., amplitude and/or frequency are increased.

If the increase generates parameters that are not desirable, for example, being outside set limits, a different electrode is selected for stimulation (618). Alternatively, the parameter values reduced (e.g., to confirm and/or recheck a previous set of stimulation parameters).

Optionally, at 620, a physician fine-tunes stimulation parameters and/or electrodes for the patient, for example, to minimize side effects.

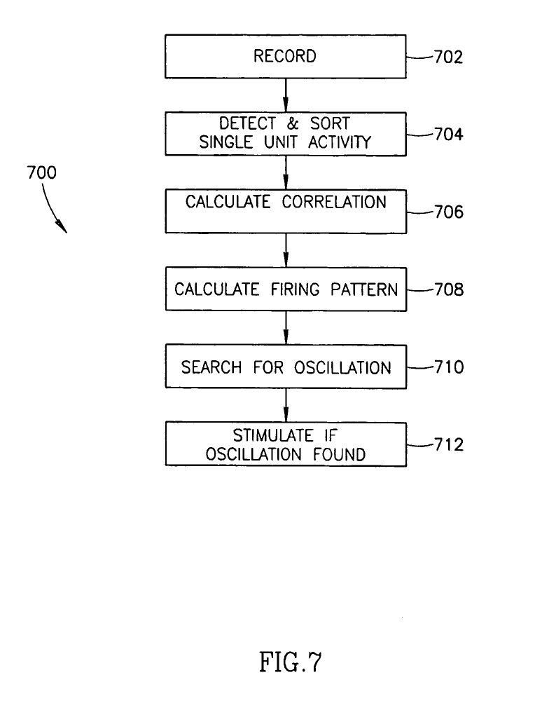

FIG. 7 is a flowchart 700 of a method of abnormal activity detection, in accordance with an exemplary embodiment of the invention. Such a method may be applied, for example, if the lead is attached to an implanted stimulator/recorder. In an exemplary embodiment of the invention, the stimulation is only applied when the patient is experiencing abnormal activity (e.g., oscillatory behavior). In particular, during sleep, no such activity exists and stimulation may be stopped.

At 702, recording from at least one sensing electrode and desirably several, is started.

At 704, the recorded signals are analyzed to detect and sort (e.g., by location) the activity of single units (e.g., single neurons). This analysis may be used to reject measurements that include signals from two or more cells or to separate the signals from the different cells.

At 706, correlation between units is determined, for example, to detect causal chains.

At 708, the firing pattern for a unit over time is calculated.

At 710, the calculated firing pattern is searched for oscillatory behavior, as opposed to burst and/or monotonous behavior.

At 712, if such oscillatory behavior is found, stimulation is applied. The checking and stimulating process may be, for example, continuous, periodic or triggered (e.g., by patient control).

Such detection of abnormal activation may also use a database of expected signals and/or their characteristics to determine if an abnormal signal is being detected and/or what stimulation is the best reply for such a signal. Alternatively or additionally, such a database or the above analysis is used to determine if a lead moved. For example, if the signals are all shifted one electrode row down, the lead probably moved and the stimulation can be shifted one row, while optionally warning the user. If however, no match can be found, stimulation is stopped until the cause is determined. Optionally, one or more electrodes, or time slots of recordings of the electrodes are reserved for use for detecting motion of the lead.

An exemplary implanted device includes a memory for recording signals and/or signal characteristics of a plurality of leads, including, for example, pure sensed values, responses to stimulation and other sensors, such as acceleration sensors. Alternatively or additionally, the device continuously and/or periodically transmits measurements to a local monitor (e.g., in a hospital or at home) or a remote monitor (e.g., at a doctor's office), or the local monitor retransmits the signals. In an exemplary embodiment of the invention, the device also generates alerts, which may be audible to the user and/or may be received by a central monitoring location, for example, if the lead appeared to have moved.

It will be appreciated that the above described multi-electrode leads and methods of deploying such electrodes may be varied in many ways, including, changing the order of acts, which acts are performed more often and which less often, the type and order of tools used and/or the particular timing sequences used. Further, the location of various elements may be switched, without exceeding the sprit of the disclosure. In addition, a multiplicity of various features, both of methods and of devices have been described. It should be appreciated that different features may be combined in different ways. In particular, not all the features shown above in a particular embodiment are necessary in every similar exemplary embodiment of the invention. Further, combinations of features from different embodiments into a single embodiment or a single feature are also considered to be within the scope of some exemplary embodiments of the invention. In addition, some of the features of the invention described herein may be adapted for use with prior art devices, in accordance with other exemplary embodiments of the invention. The particular geometric forms and measurements used to illustrate the invention should not be considered limiting the invention in its broadest aspect to only those forms. Although some limitations are described only as method or apparatus limitations, the scope of the invention also includes apparatus designed to carry out the methods and methods of using the apparatus. In particular, the above described methods are typically implemented using hardware, software and/or firmware that is suitably designed and/or programmed.

Also within the scope of the invention are surgical kits, for example, kits that include leads and stylets. Optionally, such kits also include instructions for use. Measurements are provided to serve only as exemplary measurements for particular cases, the exact measurements applied will vary depending on the application. When used in the following claims, the terms “comprises”, “comprising”, “includes”, “including” or the like mean “including but not limited to”.

It will be appreciated by a person skilled in the art that the present invention is not limited by what has thus far been described. Rather, the scope of the present invention is limited only by the following claims.

1. A multi-electrode lead for neural applications in the brain, comprising:
an elongate body having a tip and an axis; and
a plurality of electrodes arranged at said tip, each of said electrodes having a limited angular sensitivity relative to said axis.

2. A lead according to claim 1, wherein said electrodes are radially separated.

3. A lead according to claim 1, wherein said electrodes are axially separated.

4. A lead according to claim 1, wherein said electrodes are single cell sensing electrodes.

5. A lead according to claim 1, wherein said electrodes are selectively extendible.

6. A multi-electrode lead, comprising:
a delivery tube adapted to be inserted into a brain and having an axis; and
a plurality of micro-electrodes which are provided through said tube, said micro-electrodes having sensing areas which define a surface, wherein said surface is not a plane perpendicular to said axis.

7. A lead according to claim 6, wherein said surface is planar and inclined to said axis.

8. A lead according to claim 6, wherein said surface is curved.

9. A lead according to claim 6, wherein said electrode tips define a sensing volume which is bounded by said surface on at least one side thereof.

10. A multi-electrode lead, comprising:
a delivery tube adapted to be inserted into a brain and having an axis; and
a plurality of micro-electrodes held together by a water soluble material and being pre-stressed to deploy by moving apart when said material dissolves.

11. A lead according to claim 10, wherein said electrodes move apart at least 200 micro meters, from each other, when they deploy.

12. A multi-electrode delivery system, comprising:
a lead body having an axis and defining at least one stimulation electrode; and
a plurality of micro-electrodes, wherein said micro-electrodes are adapted to be delivered along said axis.

13. A delivery system according to claim 12, wherein said micro-electrodes are provided through a channel of said lead.

14. A delivery system according to claim 12, wherein said micro-electrodes are provided through a guide tube that encloses said lead.

15. A delivery system according to claim 12, wherein at least some of said micro-electrodes are held together by a water soluble material and are pre-stressed to deploy by moving apart when said material dissolves.

16. A delivery system according to claim 12, wherein said at least one stimulation electrode comprises a plurality of axially spaced stimulation electrodes.

17. Apparatus for locating a location in the brain, comprising:
means for detecting electrical signals from a plurality of locations in the brain;
means for detecting correlation between the detected signals; and
computing means for determining said location based on said correlation.

18. Apparatus according to claim 17, wherein said means for detecting comprises means for simultaneously detecting.

19. Apparatus according to claim 17, wherein said means for detecting comprises a plurality of implanted spaced apart electrodes adapted to ensure straddling of said location.

20. A method of implanting an electrode in a brain, comprising:
advancing a multi-electrode lead past an estimated location of interest in the brain;
sensing signals from electrodes of said lead; and
analyzing said signals to generate a more exact estimate of said location.

21. A method according to claim 20, comprising selectively stimulating at said more exact estimate of location, to effect a treatment of a patient.

22. A method of locating a position of a functional location in a brain, comprising:
detecting signals from a plurality of locations in a brain, which locations have a known physical positional relationship; and
correlating a behavior of said signals to determine a position of a specific functional location of the brain.

23. A method according to claim 22, comprising:
assuming a function of said brain at a position; and
using said correlation to verify said function.

24. A method according to claim 22, wherein said plurality of locations comprises functional locations.

25. A method according to claim 22, wherein said plurality of locations comprises physical locations.

26. A method according to claim 22, wherein correlating comprises comparing to a database of functional signals.

27. A method according to claim 22, wherein correlating comprises matching a spatial pattern of said signals to an expected pattern.

28. A method according to claim 22, wherein correlating comprises matching between signals of different locations.

29. A method according to claim 22, wherein detecting comprises detecting simultaneously.

30. A method according to claim 22, wherein correlating comprises detecting a response of a signal at at least one location to stimulation at a second location.

31. A method according to claim 22, wherein said signals are single cell signals and comprising setting parameters for stimulation of the brain responsive to said detected signals.

32. A cranial tap, comprising:
a body having an aperture therein, wherein said body is adapted to be attached to a hole in a skull and adapted to have mounted thereon a guide for an intra-cranial electrode lead that passes said aperture; and
a cap adapted to seal said aperture of said body after insertion of said body into said hole.

33. A method of abnormal activity detection, comprising:
(a) inserting a plurality of electrodes into a brain region;
(b) receiving signals from said plurality of electrodes;
(c) analyzing said signals to determine an abnormal signal; and
(d) applying stimulation to a selected part of said brain region, responsive to said determined abnormal signal.

34. A method according to claim 33, wherein inserting comprising implanting said electrodes as part of an implantable brain stimulation system.

35. A method according to claim 33, wherein said electrodes are mounted on a single lead.

36. A method according to claim 33, wherein said abnormal signal comprises an oscillatory signal and wherein said applying comprises applying if said oscillatory signal is detected.

37. A method according to claim 33, wherein said abnormal signal comprises a spatial shifting of the signal and wherein said applying comprises not applying if shifting of said signal is detected.

38. A method according to claim 33, wherein analyzing comprises comparing said received signals to at least one expected signal.

39. A method according to claim 33, wherein analyzing comprises comparing said received signals to at least one expected signal characteristic.

40. A method according to claim 33, wherein analyzing comprises comparing said received signals to a database.

41. A method according to claim 33, wherein applying comprises selecting a stimulation to apply responsive to said determined signal.

42. A method according to claim 33, wherein receiving comprises sorting by location.

43. A method according to claim 33, wherein analyzing comprises correlating signals from different electrodes.

44. A method according to claim 33, wherein analyzing comprises detecting a local field potential.

45. A method according to claim 33, wherein analyzing comprises detecting a causal chain between different electrodes.

46. A method according to claim 33, wherein analyzing comprises detecting a causal chain between different electrodes.

47. A method according to claim 33, wherein analyzing comprises analyzing a pattern over time in at least one electrode.

48. A method according to claim 33, wherein receiving comprises receiving in a dedicated abnormal detection timeslot.

49. A method according to claim 33, wherein receiving comprises receiving on a dedicated abnormal detection electrode.

50. A method according to claim 33, comprising generating an alert to a user on detection of abnormal activity.

51. A method according to claim 34, wherein said implantable brain stimulation system is programmable and applies a stimulation responsive to a response of a brain to a previous stimulation.

52. A method according to claim 34, wherein said implantable brain stimulation system is programmable and reconfigures itself to apply different stimulation configurations in response to measurement of brain activity.

53. An implantable brain stimulation system, comprising:
at least one electrode;
a sensing circuit attached to at least one of said at least one electrode and adapted for sensing brain activity;
a stimulation circuitry attached to at least one of said at least one electrode and adapted to apply a brain stimulation; and
a controller uses said sensed brain activity to decide on stimulation.

54. A system according to claim 53, wherein said controller reconfigures an internal database in response to sensed brain activity.
